(19) United States
(12) Patent Application Publication
(10) Pub. No.: US 2005/0227941 A1
(43) Pub. Date: Oct. 13, 2005

(54) Sequestration of ass in the periphery in the absence of immunomodulating agent as a therapeutic approach for the treatment or prevention of beta-amyloid related diseases

(76) Inventors: Karen Duff, Village Apt. 6J, NY (US); Yasuji Matsuoka, Melvern Dr., MD (US)

Correspondence Address: MORGAN & FINNEGAN, L.L.P., 3 WORLD FINANCIAL CENTER, NEW YORK, NY, 10281-2101, US

(21) Appl. No.: 10/499,125

(22) PCT Filed: Dec. 17, 2002
(86) PCT No.: PCT/US02/40212

§ 371 Date: May 24, 2005

Related U.S. Application Data
(60) Provisional application No. 60/341,145, filed on Dec. 17, 2001.

(51) Int. Cl. G01N 33/53; G01N 33/537; G01N 33/543; A61K 31/739; A61K 31/655

(52) U.S. Cl. 514/54; 514/150; 435/7.92

(57) ABSTRACT

The present invention describes a method of administering an Aβ-binding agent or drug which has affinity for amyloid beta (Aβ) in the periphery (blood) and reducing Aβ levels in the brain without the need for the agent or drug to enter the brain itself. The Aβ-binding agents utilized in the methods of the invention are preferably non-immunomodulating agents (e.g., antigenic peptides or antibodies) and bind to Aβ in the periphery, or blood. Such compounds do not significantly cross the blood/brain barrier, and yet they lower amyloid (Aβ) levels in the brain, thereby serving as safer, therapeutic and prophylactic treatments against diseases associated with Aβ in the brain, e.g., Alzheimer's Disease and amyloid angiopathy, as well as against other AD-related amyloidoses.

The work described herein is supported in part by a grant from the National Institutes of Health, National Institute of Aging, AG17585.

### FIELD OF THE INVENTION

The present invention relates to improved drug delivery methods and the discovery and development of novel compounds and drugs for the treatment and prevention of neurological diseases and disorders associated with β-amyloid, such as Alzheimer's disease, β-amyloid related problems in Down's syndrome and vascular dementia (cerebral amyloid angiopathy) and other amyloidosis diseases. The invention further relates to diagnostic and screening methods for determining or identifying the aforementioned diseases and disorders associated with β-amyloid in patients.

### BACKGROUND OF THE INVENTION

Alzheimer's Disease (AD) is the most common cause of chronic dementia, with approximately two million people in the United States having the disease. The histopathologic lesions of Alzheimer's disease (i.e., neuritic amyloid plaques, neurofibrillary degeneration, and granulovascular neuronal degeneration) are found in the brains of elderly people with Alzheimer's dementia.

It is estimated that ten percent of individuals older than 65 years of age have mild to severe dementia. The number of such lesions correlates with the degree of intellectual deterioration. This high prevalence, combined with the rate of growth of the elderly segment of the population, make dementia (and particularly AD) one of the most important of the present-day public health concerns.

An invariant feature of Alzheimer's disease (and AD in Down's syndrome) is the deposition of the small, i.e., approximately 40 to 42 residues, amyloid beta (also referred to as Aβ or Abeta herein) peptide as insoluble β-amyloid plaque in the brain parenchyma. (G. G. Glenner et al., 1984, Appl. Pathol., 2(6):357-69; G. G. Glenner et al., 1984, Biochem Biophys Res Commun., 120(3):885-90; G. G. Glenner et al., 1984, Biochem Biophys Res Commun., 122(3):1131-5). In cerebral amyloid angiopathy, Aβ is deposited in the vasculature. Aβ is generated by proteolysis of the approximately 100 kDa amyloid precursor protein (APP), a broadly expressed type-1 transmembrane protein that is found primarily in the trans Golgi network (TGN) and at the cell surface (reviewed in B. De Strooper and W. Annaert, 2000, “Proteolytic processing and cell biological functions of the amyloid precursor protein.” J. Cell. Sci., 113(Pt 11)(7):1857-1870). The β-amyloid precursor protein APP is further described in D. J. Selkoe et al., 1988, Proc. Natl. Acad. Sci. USA., 85(19):7341-7345; R. E. Tanzi et al., 1988, Nature, 331(6156):528-530; and E. Levy et al., 1990, Science, 248(4959):1124-1126.

Amyloid plaques containing Abeta (Aβ) peptides are one of the most significant pathological features of the human Alzheimer's disease brain. Drugs that reduce brain Aβ levels, or remove plaques, are considered to be the most likely to be effective in the treatment or prevention of AD. To date, treatments for AD have focused on the use of anti-Aβ antibodies or peptides which evoke the production of anti-Aβ antibodies, i.e., vaccine therapy. (see, for example, D. Schenk et al., 1999, Nature, 400(6740):173-177 and F. Bard et al., 2000, Nat Med, 6(8):916-919).

The presumed mode of action for such antibody, or immunomodulatory, treatments is in the clearance of Aβ directly from the brain due to the entry of antibodies into the brain. A side effect of vaccination is an increase of peripheral Aβ levels as levels of Aβ decrease in the brain, thus resulting in a little understood change in the dynamics between the two systems, brain and periphery.

Vaccination involving anti-Aβ antibodies is a potentially ineffective and possibly even dangerous approach for treatment of AD patients, particularly the elderly who lack (or have less) immune responsiveness, due to the risk of provoking autoimmune diseases.

Clearance of Aβ from the brain has been reported using immunization of Aβ peptides or passive immunization with anti-Aβ antibodies (D. Schenk et al., 1999, Nature, 400(6740):173-177 and F. Bard et al., 2000, Nature Med., 6(8):916-919). The proposed mechanism for this clearance is microglial phagocytosis carried out by brain immune cells, i.e., microglia, that have been activated by elicited or injected anti-Aβ antibodies.

A goal in the field of therapy and prevention of AD and amyloid-related diseases is the discovery and development of new drugs that are effective due to their mode of action in the periphery, rather than in the brain, thus obviating the need to enter the brain itself and overcoming the problems encountered in efficient dosage and effectiveness due to the blood/brain barrier. The present invention satisfies this goal by providing new methods of treatment and prevention for AD and other amyloid-related diseases, and by describing new types of drugs and compounds that serve to treat or prevent disease via the blood.

### SUMMARY OF THE INVENTION

The present invention provides methods and compounds (also termed drugs, substances, reagents, or agents, preferably bioactive agents) employed therein for sequestering Aβ in the blood, or blood components, such as plasma, i.e., the periphery, thereby reducing Aβ levels in the brain for treatment or prevention of beta-amyloid related diseases. The compounds of the invention have an affinity for (i.e., “sequester”) Aβ, and bind to and sequester Aβ in the blood, or periphery, e.g., plasma, without needing to enter the brain itself. According to this invention, such compounds do not (and do not need to) cross the blood/brain barrier, and yet they significantly lower amyloid (Aβ) levels in the brain. Such compounds have been shown in animal models of disease, e.g., a transgenic AD mouse model, to lower Aβ levels in the brain by sequestering Aβ in the periphery, e.g., plasma. That the invention provides a method and drugs used therein which obviate the need for a drug to enter the brain itself, while still significantly lowering amyloid (Aβ) levels in the brain, offers a great improvement over drugs that currently must enter the brain to have an effect on Aβ levels in the brain.

Thus, it is also an aspect of the present invention to provide a method of treating or preventing AD comprising administering to an individual in need thereof a compound or drug having an affinity for Aβ, which binds to Aβ in the periphery, wherein such a compound or drug, preferably a non-immune related compound or drug, and also preferably, an agent other than an antibody or an immunomodulating agent, sequesters Aβ in the periphery and leads to a reduction in Aβ levels in the brain. For delivery to the periphery, such Aβ-binding compounds are preferably introduced intravenously or subcutaneously; however, any method of introducing the compound into the blood stream (including via pumps) is acceptable and suitable in accordance with this invention.

It is another aspect of the present invention to provide a method of reducing amyloid (or soluble/insoluble Aβ) levels in the brain of a patient undergoing treatment by obviating the need to introduce an Aβ-binding drug or compound directly, or indirectly, into the brain. According to the invention, the effectiveness of the method in which the Aβ-binding drug sequesters Aβ in the bloodstream and removes it from the brain is at least as high as a vaccine approach involving the production of antibodies that cross the blood/brain barrier, enter into the brain, and act in the brain.

It is yet a further aspect of the present invention to provide a method for diagnosing, screening, or monitoring treatment of diseases involving β-amyloid or amyloidoses, such as AD, comprising determining an elevation of Aβ levels in the periphery, e.g., plasma, particularly at a time, or series of times, following treatment. In accordance with this aspect of the invention, the elevation of Aβ levels in the periphery, e.g., plasma, serves as a diagnostic marker of diseases involving β-amyloid, particularly, AD.

Further aspects, features and advantages of the present invention will be apparent when considered in connection with the further disclosure of the invention hereinbelow.

### DESCRIPTION OF THE INVENTION

In one of its aspects, the present invention describes compounds (drugs) which have an affinity for, i.e., “sequester”, Aβ in the blood, or blood components, e.g., plasma, (periphery) and which reduce Aβ levels in the brain without the need of the compounds (e.g., drugs or bioactive agents) to enter the brain itself. Such compounds sequester Aβ in the periphery and alter the periphery/brain dynamics so as to reduce Aβ in the brain by virtue of their effective sequestration of Aβ in the periphery.

According to this invention, such compounds are preferably brain impermeable and essentially do not (and do not need to) cross the blood/brain barrier following administration or introduction into a recipient, and yet they significantly lower amyloid (Aβ) levels in the brain. Also in accordance with this invention, such compounds have been shown in animal models of disease, e.g., AD mouse models, to lower Aβ levels in the brain by sequestering Aβ in the periphery, e.g., plasma. (Example 1). It is thus an aspect of the invention that the Aβ-binding agent, drug, compound, and the like, effectively sequesters Aβ in the periphery following administration in the periphery. Preferably, greater than about 50% of the Aβ-binding agent, drug or compound remains in the periphery versus the brain following administration in the periphery. More preferably, about 90% or more of the Aβ-binding agent, drug or compound remains in the periphery versus the brain following administration in the periphery

In addition, the finding of elevated Aβ in the periphery, particularly in plasma, preferably in conjunction with the administration of agents that bind Aβ and sequester Aβ in the periphery, can serve as a diagnostic marker of β-amyloid-related diseases, especially, AD. The elevation of levels of Aβ in the periphery can further serve as a means of monitoring the effectiveness of treatment of a disease involving Aβ, particularly with a drug or agent that binds and sequesters Aβ in the periphery, thereby leading to its elevation in the periphery. According to this embodiment of the invention, an elevation of Aβ in the periphery reflects an amount of Aβ that is increased relative to that found in normal individuals, such as in plasma, or a base level of Aβ, e.g., in plasma, in individuals who serve as controls. Determining and/or measuring levels of Aβ can be performed using routine techniques as known in the art, such as radioimmunoassays (RIAs), nonradioactive immunoassays, such as enzyme linked immunoassays (ELISAs), western blotting, dot blotting, mass spectrometry, etc.

Without wishing to be bound by theory, it is proposed that microglial phagocytosis is not necessary for Aβ clearance from the brain in accordance with the present invention. Instead, sequestration in the blood, or blood component, such as plasma, i.e., the periphery, in the absence of an immune modulating agent, by suitable Aβ-binding compounds that are not Aβ peptides or their derivative antibodies, serves to reduce Aβ levels in the brain and to alter the central nervous system (CNS)/periphery dynamics leading to reduction of Aβ in the brain. As used herein, the terms immune modulating agent, and immune related agent, refer to an anti-Aβ antibody or a peptide against some region of Abeta or APP that evokes the production of antibodies, which recognize an Abeta region or APP.

As a consequence of the sequestration of Aβ in the periphery according to this invention, higher levels of Aβ are predicted to be found in the blood (e.g., plasma)/periphery), (see Example 1), because Aβ is sequestered in the blood/periphery by the Aβ-binding compounds. It is also to be understood that the removal of bound Aβ/binding agents by cellular clearance mechanisms may effectively reduce the levels of peripheral Aβ seen following administration of the sequestering agent. The important effect of the methods and reagents of the present invention is that the levels of Aβ found in the brain as a result of keeping Aβ sequestered in the periphery are reduced, which is advantageous for the therapeutic effect of the method and compounds of the present invention.

The invention also allows for determining or monitoring a drug's effectiveness by monitoring Aβ levels in the periphery, such as in the plasma, instead of, or in addition to, brain Aβ levels. (see, e.g., Example 1). Methods of monitoring Aβ levels in an individual undergoing drug treatment or therapy for amyloid related diseases involve determining the levels of Aβ in the individual's peripheral body fluid sample, e.g., plasma, at one or more time intervals following treatment or therapy involving an Aβ binding agent that sequesters Aβ in the periphery. For example, an individual can be monitored at about 1-25 hours, preferably at about 2-10 hours following administration of the Aβ binding agent, or at varying time intervals therebetween, to determine if Aβ levels are elevated. Such monitoring methods are particularly useful for determining if a given drug treatment is beneficial, or to determine if doses of a drug or a drug combination should be modified or adjusted during the course of treatment. In such methods, it is preferred to use the individual's pretreatment levels of Aβ in the periphery to compare and assess treatment and post-treatment levels of Aβ in the periphery. Controls can also include peripheral levels of Aβ in disease-free (or dementia-free) individuals, as well as peripheral levels of Aβ in Individuals having an amyloid related disease, e.g., AD. Human plasma and cerebrospinal fluid levels of amyloid beta proteins, particularly, Aβ40 and Aβ42, have been reported (see, e.g., Mehta et al., 2000, Arch. Neurol., 57:100-105).

According to this invention, Aβ levels can be measured in an individual's body fluid sample, such as blood, serum, or plasma, using conventionally known assays that detect Aβ, for example, radioisotopic immunoassays or non-isotopic immunoassays, e.g., fluorescent immunoassays, chemiluminescent immunoassays and enzymatic immunoassays, such as an enzyme linked immunoassay (ELISA), as are commercially available, known and practiced in the art, for example, Beta-amyloid (Abeta) [1-40] Immunoassay (Biosource, Camarillo, Calif.; Cat. No. KHB3481); Beta-amyloid (Abeta) [1-42] Immunoassay (Biosource, Camarillo, Calif.; Cat. No. KHB3441); and Human Amyloid beta (1-40) Immunoassay (IBL, Fujioka, Gunma, Japan; Cat. No. 17713).

Typically, an ELISA assay initially involves preparing, obtaining, or employing an antibody specific to Aβ, preferably a monoclonal antibody. In addition, a reporter antibody is used. In some ELISA protocols, the reporter antibody recognizes and binds to the anti-Aβ-specific monoclonal antibody. To the reporter antibody is attached a detectable reagent such as a radioactive isotope, a fluorescent moiety, a chemiluminescent moiety, or, in an ELISA, an enzyme, such as horseradish peroxidase or alkaline phosphatase.

As is appreciated by those skilled in the art, ELISAs can be performed in a number of assay formats. In one ELISA format, a host sample, e.g., a patient body fluid sample, is incubated on a solid support, e.g., the wells of a microtiter plate, or a polystyrene dish, to which the proteins in the sample can bind. Any free protein binding sites on the dish are then blocked by incubating with a non-specific protein such as bovine serum albumin. The monoclonal antibody is then added to the solid support, e.g., the wells or the dish, and allowed to incubate. During the Incubation time, the monoclonal antibodies attach to any Aβ polypeptides or peptides that have attached to the polystyrene dish.

All unbound monoclonal antibody is washed away using an appropriate buffer solution. The reporter antibody, e.g., linked to horseradish peroxidase, is added to the support, thereby resulting in the binding of the reporter antibody to any monoclonal antibody which has bound to Aβ present in the sample. Unattached reporter antibody is then washed away. Peroxidase substrate is added to the support and the amount of color developed in a given time period provides a measurement of the amount of Aβ that is present in a given volume of individual or patient sample when compared to a standard curve.

In another ELISA format, antibody specific for a particular analyte is attached to the solid support, i.e., the wells of a microtiter plate or a polystyrene dish, and a sample containing analyte is added to the substrate. Detectable reporter antibodies, which bind to the analyte that has bound to the capture antibodies on the support, are then added, after the appropriate incubations and washings, and analyte-antibody complexes are detected and quantified.

The present invention also embraces a sandwich type ELISA immunoassay typically performed using microliter plates. A capture antibody, that can be polyclonal or monoclonal, preferably a monoclonal antibody, that specifically recognizes an epitope in the Aβ peptide is used, along with a labeled detector antibody, e.g., an alkaline phosphatase-labeled antibody, or a horse radish peroxidase-labeled antibody, preferably a monoclonal antibody. The detector antibody also specifically recognizes an epitope in Aβ. Preferably also, the capture antibody does not inhibit binding to Aβ. The production of both polyclonal and monoclonal antibodies, particularly monoclonal antibodies that are specific for Aβ, is performed using techniques and protocols that are conventionally known and practiced in the art.

In a particular embodiment according to this invention, a capture anti-Aβ antibody of the assay method is immobilized on the interior surface of the wells of the microtiter plate. To perform the assay, an appropriate volume of sample is incubated in the wells to allow binding of the antigen by the capture antibody. The immobilized antigen is then exposed to the labeled detector antibody. Addition of substrate to the wells, if the detectable label is alkaline phosphatase, for example, allows the catalysis of a chromogen, i.e., para-nitrophenylphosphate (pNPP), if the label is alkaline phosphatase, into a colored product. The intensity of the colored product is proportional to the amount of Aβ that is bound to the microtiter plate.

Standards are used to allow accurate quantitative determinations of Aβ in the samples undergoing analysis. A microtiter plate reader simultaneously measures the absorbance of the colored product in the standard and the sample wells. Correlating the absorbance values of samples with the standards run in parallel in the assay allows the determination of the levels of Aβ in the sample. Samples are assigned a quantitative value of Aβ in nanograms per milliliter (ng/ml) of blood, serum, plasma, or other body fluid.

The present invention provides a significant advantage to the treatment and prevention of AD and amyloid-related diseases in that drugs and active compounds according to this invention are not required to cross the blood/brain barrier to exert their effect. Having to cross the blood/brain barrier is an enormous obstacle to developing effective drugs for use in the brain. This invention overcomes this obstacle. One of the major differences between this method and others is that it uses a non-antibody compound, or a compound that is not related to an antibody, to achieve the sequestration of Aβ, and that this sequestration has its primary effect in the periphery. A consequence of the method and the compounds utilized therein is a decrease in Aβ in the brain.

In addition, a second advantage of this invention is that neither Aβ peptides, nor anti-Aβ antibodies, is administered to a host, thus negating the risk of an adverse immune response, or the lack of an effective immune response. For AD and amyloid angiopathy, this method preferably involves the use of Aβ-binding compounds and drugs of the invention, more preferably formulated as pharmaceutically acceptable compositions as described herein.

The method further comprises the administration of an amyloid beta (Aβ)-binding compound, agent, or drug in the periphery of an individual in need thereof, wherein the compound sequesters Aβ in the periphery and concomitantly decreases Aβ levels in the brain of the individual undergoing treatment. According to the method of the present invention, the need to introduce an agent or drug directly into the brain, or to have the drug cross the blood/brain barrier is obviated. Also according to this method, brain Aβ levels are reduced, due to the effects of the Aβ-binding drugs and compounds described herein on Aβ in the periphery.

The method and Aβ-binding compounds and agents used therein in accordance with the present invention are suitable for the treatment, both prophylactic and therapeutic, of neurological diseases and disorders associated with β-amyloid, such as Alzheimer's disease, β-amyloid related problems in Down's syndrome and vascular dementia (cerebral amyloid angiopathy) (A. J. Rozemuller et al., 1993, Am. J. Pathol., 142(5):1449-1457) and other amyloidosis diseases. The method involving peripheral sequestration of disease associated agents, e.g., peptides, or proteins or aggregates thereof, by non-immunomodulating agents that bind to such disease associated agents in the blood/periphery in accordance with the present invention, are also useful in the treatment or prevention of other cortical or vascular amyloidoses, including those caused by cystatin C (ACys), prion protein (AScr), transthyretin (ATTR), gelsolin (AGel), and Amyloid ABri (or A-WD) (see, M. Yamada, 2000, “Cerebral amyloid angiopathy: an overview”, Neuropathology, 20(1):8-22). Cortical or vascular amyloidoses are very similar in etiology to AD.

In accordance with the present invention, the method of delivering Aβ-binding drugs to the periphery has been shown to be at least as effective as the vaccine approach in transgenic mouse models, if not more so. Indeed, the use of non-toxic, non-immune related compounds and drugs can overcome adverse immune responses that are frequently associated with the use of brain-directed immunovaccines. Prior to the present invention, the treatment of brain amyloidosis by administering non-immune related Aβ-binding agents in the periphery and sequestering or “locking away” Aβ in the blood, or periphery, has not been shown.

Suitable compounds that can be employed in the method of this invention include, but are not limited to, small molecules, e.g., peptides, proteins; biologic agents; and drugs that have an affinity for Aβ and bind Aβ in the periphery. Such compounds, molecules, agents and drugs have an Aβ-binding domain that physically binds to and locks away Aβ in the periphery. The compound, molecule, agent or drug can bind to or have affinity for a variety of Aβ peptides, e.g., Aβ peptides derived from Aβ P; Aβ peptides of different fragment lengths, e.g., Aβ40 or Aβ42, and the like. In addition, the compound, molecule, agent or drug can bind to or have affinity for any portion of an Aβ peptide, e.g., the N- or C-terminus, or other regions of the molecule. Non-immune related and/or non-immunomodulatory compounds or drugs are preferred. Most preferably the compounds are nontoxic and well tolerated following their use in the treatment and prevention methods.

An advantage of the use of molecules other than immune related compounds, such as antibodies, for peripheral sequestration of Abeta (Aβ) is that non-antibody related drugs can be manipulated more easily than antibodies. For example, sequestering compounds can be modified to be metabolized faster by the addition of certain chemical structures, as known and practiced in the art. For example, sequestering compounds can be modified by the addition of side chain(s) which can modulate metabolism. Such chemical modification of the non-antibody Aβ-binding and sequestering compounds can improve their efficacy and reduce toxicity and/or potentially adverse side effects. The derivative of Congo Red, an Aβ-imaging agent, as described herein, is particularly suitable for chemical derivatization or modification.

Nonlimiting examples of such Aβ-binding compounds include compounds having an affinity for Aβ, particularly, cortigangliosides, such as GM1, the actin-regulating molecule gelsolin, particularly, the extracellular Aβ-binding domain of gelsolin, and Aβ staining molecules, such as derivatives of Congo Red, e.g., [1,4-bis(3-carboxy-4-hydroxyphenylethenyl)-benzene and 5,5′-[(1,1′ biphenyl)-4,4′-diylbis(azo)]bis [2-hydroxybenzoic acid]disodium salt (chrysamine-G or CG), as described in U.S. Pat. No. 6,133,259 and WO 96/34853. A preferred Aβ staining molecule is the Aβ staining dye compound Chrysamine-G, as described in U.S. Pat. No. 6,133,259 and WO 96/34853. (Example 2). Other nonlimiting examples of Aβ binding agents that are suitable for use in the methods of this invention include Aβ imaging agents (e.g., Klunk et al., 1995, “Chrysamine-G binding to Alzheimer and control brain: autopsy study of a new amyloid probe”, Neurobiol. Aging, 16: 541-548), β-sheet breakers (e.g., Bohrmann et al., 2000, “Self-assembly of beta-amyloid 42 is retarded by small molecular ligands at the stage of structural intermediates”, J. Struct. Biol., 130:232-246), β-sheet formation inhibitors (e.g., Findeis et al., 1999, “Modified-peptide inhibitors of amyloid beta-peptide polymerization”, Biochemistry, 38:6791-6800), and the like, are encompassed for use in the present invention.

The Aβ-binding compounds according to the invention can be incorporated into pharmaceutical formulations, or pharmaceutical compositions, preferably physiologically acceptable compositions, according to known methods, such as by admixture with a pharmaceutically acceptable carrier, diluent, or excipient. One or more Aβ-binding compounds or drugs comprise the pharmaceutical compositions and are formulated as active ingredients in the compositions in a therapeutic or prophylactic amount.

The pharmaceutically, or physiologically, acceptable carrier, diluent, or excipient can be any compatible nontoxic substance suitable to deliver the compound to a host or recipient. Sterile water, alcohol, fats, waxes and inert solids may be used as carriers. In addition, pharmaceutically acceptable adjuvants, buffering agents, dispersing agents, and the like, may also be incorporated into the pharmaceutical compositions. The preparation of pharmaceutical compositions comprising active agents is well described in the scientific and medical literature. Examples of methods of formulation, and carriers, etc. may be found in the latest edition of Remington's Pharmaceutical Sciences, 18th Ed., 1990, Mack Publishing Co, Easton, Pa.

To formulate a pharmaceutically acceptable composition suitable for effective administration, preferably in vivo, or even ex vivo, such compositions will contain an effective amount of the active compound, biomolecule, agent or drug. Pharmaceutical compositions of the present invention are administered to an individual in amounts effective to treat or prevent AD, amyloid angiopathy, or other Aβ-associated diseases or conditions. The effective amount may vary according to a variety of factors, such as an individual's physical condition, weight, sex and age. Other factors include the mode and route of administration. These factors are realized and understood by the skilled practitioner and are routinely taken into account when administering a therapeutic agent to an individual.

Pharmaceutical compositions suitable for use in the present invention include compositions wherein the active ingredients are contained in an effective and sufficient amount to directly bind Aβ in the periphery, sequester it there, and reduce the Aβ levels in the brain. The determination of an effective dose is well within the capability of the skilled practitioner in the art. The therapeutically effective dose can be estimated initially either in cell culture assays, or in animal models, usually mice, rabbits, dogs, pigs, rats, monkeys, or guinea pigs. The animal model is also used to achieve a desirable concentration range and route of administration. Such information can then be used to determine useful doses and routes for administration in humans.

A therapeutically effective dose refers to that amount of the Aβ-binding compound or drug which ameliorates, reduces, or eliminates the symptoms or condition. In accordance with this invention, the effective dose is preferably that which lowers, reduces, or eliminates levels of Aβ, or buildup of Aβ, in the brain, while binding to and “locking up” Aβ in the periphery. The exact dosage is chosen in view of the patient to be treated, the route of administration, the severity of disease, and the like.

The concentration of the Aβ-binding drug, compound or bioactive agent in the pharmaceutical carrier may vary, e.g., from less than about 0.1% by weight of the pharmaceutical composition to about 20% by weight, or greater. As a nonlimiting example, a typical pharmaceutical composition for intramuscular administration would be formulated to contain one to four milliliters (ml) of sterile buffered water and one microgram (μg) to one milligram (mg) of the Aβ-binding drug or compound of the present invention. A typical composition for intravenous infusion could be formulated to contain, for example, 100 to 500 ml of sterile buffered water or Ringer's solution and about 1 to 100 mg of the Aβ-binding drug or compound.

The daily dosage of the pharmaceutical, or physiologically acceptable, products may be varied over a wide range, for example, from about 0.01 to 1,000 mg per adult human/per day. An effective amount of the drug is ordinarily supplied at a dosage level of from about 0.0001 mg/kg to about 100 mg/kg of body weight per day. The range is more particularly from about 0.001 mg/kg to 10 mg/kg of body weight per day. Even more particularly, the range varies from about 0.05 to about 1 mg/kg. Of course, it will be understood by the skilled practitioner that the dosage level will vary depending upon the potency or effectiveness of a particular compound, or combination of compounds, and that certain compounds will be more potent or effective than others.

In addition, the dosage level will vary depending upon the bioavailability of the compound. The more bioavailable and potent the compound, the less amount of the compound will need to be administered through any delivery route, including, but not limited to, oral delivery. The dosages of the Aβ-binding compounds are adjusted, if combined, in order to achieve desired effects. On the other hand, dosages of the various Aβ binding agents or compounds may be independently optimized and combined to achieve a synergistic result, wherein the pathology is reduced more than it would be if one single agent or compound were used alone.

The pharmaceutical compositions may be provided to an individual in need of therapeutic treatment by a variety of routes, such as; for example, subcutaneous, topical, oral, intraperitoneal, intradermal, intravenous, intranasal, rectal, intramuscular, and within the pleural cavity. Administration of pharmaceutical compositions is accomplished orally or parenterally. More specifically, methods of parenteral delivery include topical, intra-arterial, intramuscular, subcutaneous, intramedullary, intrathecal, intraventricular, intravenous, intraperitoneal, intranasal administration, or via the pleural cavity. In addition, the compounds according to the invention can be delivered via one or more routes of administration through the use of pumps.

Also embraced are transdermal modes of delivery, such as patches and the like, with or without a suitable permeation enhancer. The methods and compositions embodied by the invention provide a means by which one or more of the Aβ-binding drugs, or medicaments, can be effectively administered in a transdermal system. Frequently, compounds having poor topical absorption, or which are required at high dosage levels, are delivered transdermally. Accordingly, a transdermal means of delivering a drug composition (often with a permeation enhancing composition) to the skin is that of the transdermal patch or a similar device as known and described in the art. Examples of such devices are disclosed in U.S. Pat. Nos. 5,146,846, 5,223,262, 4,820,724, 4,379,454 and 4,956,171. The transdermal mode of storing and delivering the compositions onto the skin and forming the active composition is convenient and well suited for the purposes of the invention.

The present invention also provides suitable topical, oral, systemic and parenteral pharmaceutical formulations for use in the methods of treatment described herein. It is to be appreciated that the compositions containing the Aβ-binding compounds can be administered in a wide variety of therapeutic dosage forms in conventional vehicles for administration. For example, the compounds can be administered in such oral dosage forms as tablets or capsules (including timed release and sustained release formulations), pills, powders, granules, elixirs, tinctures, solutions, suspensions, syrups and emulsions, or by injection. Likewise, the therapeutic compounds may also be administered in intravenous (both bolus and infusion), intraperitoneal, subcutaneous, topical (with or without occlusion), or intramuscular form, all using forms well known to those of ordinary skill in the pharmaceutical arts. The preferred mode of delivery for the Aβ-binding compounds according to the present invention is intravenous.

For topical administration, the compositions of the present invention may be formulated in oil, water, or combinations thereof. Preferred is a dermatologically acceptable formulation comprising an oil-in-water emulsion. Examples of other dermatologically acceptable vehicle formulations of the present invention include, but are not limited to, any suitable nontoxic or pharmaceutically acceptable topical carrier, such as a solution, suspension, emulsion, lotion, ointment, cream, gel, plaster, patch, film, tape or dressing preparation, all of which are well-known to those skilled in the art of topical skin formulations and preparations.

The pharmaceutical compositions of the present invention can be administered for therapeutic and/or for prophylactic purposes of treating diseases, pathologies, or conditions related to the increase in Aβ levels, or the deposition of Aβ in the brain, for example, AD and amyloid angiopathy. Prophylactic treatment is preferred, although therapeutic treatment is also efficacious. For prophylactic applications, the pharmaceutical compositions of this invention are administered to an individual who is susceptible to, or prone to, the disease, pathology, or condition. Such individuals can be identified by genetic screening and/or clinical analysis, such as is described in the medical literature (see, e.g., Goate, 1991, Nature, 349:704-706 and E. H. Corder et al., 1993, Science, 261(5123):921-923). In such cases, the pharmaceutical compositions bind to or sequester AD in the periphery at a symptomatically early stage, thus preferably preventing either the initial stages of, or the severity of, disease progression. Furthermore, prophylactic treatment can be applied to any individual wishing to undertake treatment, regardless of their susceptibility.

In therapeutic applications, the pharmaceutical compositions of this invention are administered to an individual in need thereof; such individuals already suffer from, or are thought to suffer from the disease, pathology, or condition. In general, a dose of an Aβ-binding compound effective for prophylactic treatment or therapy is the same as that for therapeutic treatment or therapy.

### EXAMPLES

The following example describes specific aspects of the invention to illustrate the invention and provides a description of the present methods for those of skill in the art. The example should not be construed as limiting the invention, as the examples merely provide specific methodology useful in the understanding and practice of the invention and its various aspects.

### Example 1

Transgenic mice that develop AD-related amyloidosis, (i.e., PS/APP mice; See, L. Holcomb et al., 1998, Nature Med., 4(1):97-100) were used for the studies described in this Example to assess how peripheral sequestration of Aβ affected brain Aβ levels. The ganglioside GM1 was utilized as an exemplary Aβ-binding compound, since GM1 is known to bind Aβ strongly, and does not appear to enter the brain. In addition, a second compound, gelsolin, which is too large to cross the blood/brain barrier (BBB), and is completely unrelated to GM1, but which is also known to bind Aβ with great avidity, was administered peripherally to confirm the universality of the mechanism.

PS/APP mice were injected every two days for two weeks either with GM1 (number of mice=6) (15 mg/kg, ip,), with gelsolin (number of mice=3) (60 μg/kg, ip), or with vehicle, (phosphate buffered saline), (number=7), into the periphery at 9 weeks of age, an age when amyloid pathology in the brain is not visible. The mice were left for 1 week without injections (the “wash-out” period) and were then sacrificed at 12 weeks of age, an age when amyloid deposition has been initiated and measurable levels of Aβ are present in the vehicle treated controls.

The levels of Aβ in the peripheral blood were tested at three time points during the drug administration period (i.e., after 1 week of injections; after two weeks of injections; and after the washout period). Data for the third time point only are shown. The levels of Aβ peptides (Aβ40 or Aβ42) in the brain and plasma were assessed by ELISA assay. All of the Aβ in the brain (including Aβ in plaques) was extracted in 70% formic acid (FA). The levels of Aβ peptides in GM1 or gelsolin treated mice were compared with those of vehicle treated controls.

The results presented in Table 1 show that there was a statistically significant (p<0.05) decrease in both Aβ40 and Aββ42 in the FA-soluble brain fraction in GM1-treated mice compared with those in control animals. This correlates with a statistically significant increase in peripheral Aβ40 and Aβ42 at the same time point. For gelsolin, there was also a significant decrease in Aβ42, which correlated with a statistically significant increase in peripheral Aβ, thus confirming that the general principle of Aβ sequestration in the periphery being linked to reduction of Aβ in the CNS holds true for very different types of compounds that have the unifying property of being able to bind Aβ in the blood.

TABLE 1
Changes of brain Abeta load [fmole/mg protein] in GM1-
and Gelsolin-treated mice compared with controls (vehicle)

| Treatment | n | Abeta40 | Abeta42 |
|---|---|---|---|
| Vehicle | 7 | 3630 ± 30 | 4100 ± 200 |
| GM1 | 6 | 2147 ± 226 | 2990 ± 410 |
| | % | 59 | 73 |
| P-value | p | 0.005** | 0.031* |
| Gelsolin | 3 | 3473 ± 673 | 2473 ± 167 |
| | % | 96 | 60 |
| P-value | p | 0.502 | 0.021* |

*results statistically significant to the 5% level (p = 0.05);
**results statistically significant to the 1% level (p = 0.001).

The results presented in Table 2 show the changes in plasma load of both Aβ40 and Aβ42 in GM1-treated mice compared with control (vehicle) animals. As can be observed, there was a significant increase in plasma Abeta levels. For GM1, both Abeta40 and Abeta42 are increased; for gelsolin, Abeta42 is increased to a greater extent than is Abeta40. Thus, the effect with gelsolin may reflect a different preference for Abeta42 over Abeta40.

TABLE 2
Changes of plasma Abeta load [fmole/ml plasma] in
GM1 treated mice compared with controls (vehicle)

| Treatment | n | Abeta40 | Abeta42 |
|---|---|---|---|
| Vehicle | 7 | 100 ± 21 | 100 ± 7 |
| GM1 | 6 | 177 ± 29 | 124 ± 11 |
| | p | 0.021** | 0.036* |
| Gelsolin | 3 | 218 ± 87 | 153 ± 35 |
| | p | 0.016** | 0.039* |

*results statistically significant to the 5% level (p = 0.05);
**results statistically significant to the 1% level (p = 0.001).

Neither GM1 nor gelsolin is known to cross into the brain from the periphery to any degree. In addition, as part of the studies related to those described in this example, GM1 was introduced directly into the brain of transgenic mice, but no change in Aβ levels was observed. Thus, the results indicate that the effects of GM1 administration in the test mice is due to the sequestration of Aβ in the periphery, thereby leading to a change in dynamics between brain and peripheral Aβ transport. This is the first time that such a result has been shown for a peripherally administered compound that is not an antibody. As such, the invention affords a significant advantage to the art by describing and promoting Aβ-binding compounds that require neither penetration of the brain nor the evocation of an immune response, which are potentially harmful and ineffective ways to modulate the risk of AD in human patients.

It will be appreciated that the use of GM1 or gelsolin in the present example is not limiting to the types of compounds considered to be suitable for use in the present invention. Indeed, in accordance with this invention, any Aβ binding molecule can have the same effect following peripheral administration, thus providing a powerful treatment and therapeutic for AD sufferers, as well as those afflicted with other amyloidoses, e.g., amyloid angiopathy.

### Example 2

In this Example, as in Example 1, transgenic mice that develop AD-related amyloidosis, (i.e., PS/APP mice; See, L. Holcomb et al., 1998, Nature Med., 4(1):97-100) were used to assess the Aβ-binding compound chrysamine G (CG) in the peripheral sequestration of Aβ according to this invention, and to determine how the peripheral sequestration of Aβ by this compound affected brain Aβ levels. CG is known to bind Aβ strongly, and is less brain permeable than GM1.

PS/APP mice at 10 weeks of age were injected once either with CG (number of mice=3, dosage: 20 mg/kg) or vehicle (phosphate buffered saline, number of mice=2) into the blood stream. Blood samples were collected prior to treatment (injection) and post-treatment at 10 minutes, 2.5 hours, 5 hours and 25 hours after injection. Blood Aβ levels were compared between pretreatment versus post-treatment at 10 minutes, 2.5, 5 and 25 hours after injection. The levels of Aβ peptides (Aβ40 or Aβ42) in the plasma were assessed by ELISA immunoassay. The levels of Aβ peptides in the periphery, i.e., plasma, of CG treated mice were compared with plasma Aβ levels in pretreatment mice at various time points.

Changes in plasma Aβ levels after injection with CG were compared with pretreatment plasma Aβ levels as shown in Table 3.

TABLE 3
Changes in plasma Aβ levels after injection with CG

| Hours | Aβ42 level |
|---|---|
| after injection | [% of pre-treatment time point] |
| 2.5 | 110 ± 8 (p = 0.232) |
| 5 | 125 ± 8 (p = 0.049) |
| 7.5 | 125 ± 7 (p = 0.034) |
| 24 | 112 ± 13 (p = 0.341) |
| 48 | 111 ± 10 (p = 0.256) |

The results presented in Table 3 show that were was a statistically significant (p<0.05) increase in Aβ, as represented by Aβ42 determination, in the plasma of the mice injected with CG at 5 and 7.5 hours after injection. Changes in plasma load of Aβ42 after injection with CG were compared to the plasma Aβ level at pretreatment time points. As can be observed, there was a significant increase in plasma Aβ levels after injection of CG.

Following a oneweek wash out period, the effect in brain AD level after continuous injection was examined. PS/APP mice were injected every day for one week into the periphery, with either CG (number of mice=3) (20 mg/kg, ip,) or vehicle (phosphate buffered saline) (number of mice=2), at 11 weeks of age, an age when amyloid pathology in the brain is not visible. The mice were sacrificed at 12 weeks of age, an age when amyloid deposition has been initiated and measurable levels of Aβ are present in the vehicle treated controls.

The levels of Aβ in the peripheral blood were tested at the end of the administration period. The levels of Aβ peptides (e.g., Aβ40 or Aβ42) in the brain and plasma were assessed by ELISA assay. All of the Aβ in the brain (including Aβ in plaques) was extracted in 70% formic acid (FA). The levels of Aβ peptides in CG treated mice were compared with those of vehicle treated controls. The results presented in Tables 4 and 5 show that there was a statistically significant (p<0.05) decrease in Aβ40 and/or Aβ42 in the FA-soluble brain fraction in CG-treated mice compared with those in control animals tested 1 week after injection. This correlates with a statistically significant increase in peripheral Aβ40 and Aβ42 at the same time point.

TABLE 4
Change of plasma Aβ levels 1 week following injection with CG

| | Plasma Aβ42 level |
|---|---|
| | [% of control] |
| Vehicle | 100 ± 9 |
| CG | 331 ± 10 |
| treated | |
| | (p = 0.0081) |

TABLE 5
Change of brain Aβ 1week following injection with CG

| | Aβ40 [fmol/ml] | Aβ42 [fmol/ml] |
|---|---|---|
| Vehicle | 926 ± 26 | 1209 ± 292 |
| CG | 541 ± 50 | 540 ± 271 |
| | (P = 0.0031) | (P = 0.2038)* |

*Because of data variation, Aβ42 was not statistically significant, p = 0.20.

The method of the present invention for determining elevated levels of Aβ in the periphery for the purposes of diagnosing, screening, or monitoring patient treatment, treatment outcome, or the course and/or severity of amyloid-related disease in an individual preferably involves a pretreatment or baseline value for assessing peripheral elevation of Aβ levels in the individual undergoing testing. In the examples presented herein, an elevation of plasma Abeta was compared by percentage pre-treatment time point of an individual animal. Similar comparative assessments of pretreatment and treatment Abeta levels can be employed for the testing of other mammals, including humans, particularly because the range of Abeta levels can be large between and among individuals. As a nonlimiting guide, a representative non-elevated level of Abeta in human plasma (e.g., periphery) is about 25%, as determined experimentally (e.g., Mehta et al., 2000, Ibid.).

The contents of all patents, patent applications, published PCT applications and articles, books, references, reference manuals and abstracts cited herein are hereby incorporated by reference in their entirety to more fully describe the state of the art to which the invention pertains.

As various changes can be made in the above-described subject matter without departing from the scope and spirit of the present invention, it is intended that all subject matter contained in the above description, or defined in the appended claims, be interpreted as descriptive and illustrative of the present invention. Many modifications and variations of the present invention are possible in light of the above teachings.

1. A method of treating amyloid beta (Aβ)-associated disease, comprising administering an amyloid beta (Aβ)-binding agent in the periphery of an individual in need thereof, wherein said agent binds to Aβ in the periphery, sequesters Aβ in the periphery and concomitantly decreases Aβ levels in the brain of the individual undergoing treatment, in the absence of immunomodulating agents.

2. The method according to claim 1, wherein the amyloid beta (Aβ)-associated disease is selected from Alzheimer's disease, β-amyloid related problems of Down's syndrome, vascular dementia (cerebral amyloid angiopathy) and amyloidosis.

3. The method according to claim 1, wherein the amyloid beta (Aβ)-binding agent is selected from the group consisting of GM1 ganglioside, gelsolin, an Aβ imaging agent, a β-sheet breaker, a β-sheet formation inhibitor and a derivative of an amyloid beta (Aβ)-staining dye.

4. The method according to claim 1, wherein the derivative of the amyloid beta (Aβ)-staining dye is 1,4-bis(3-carboxy-4-hydroxyphenylethenyl)-benzene and 5,5′-{(1,1′biphenyl)-4,4′-diylbis(azo)}bis {2-hydroxybenzoic acid}disodium salt (chrysamine-G).

5. The method according to claim 1, wherein amyloid beta (Aβ)-binding agent is virtually brain impermeable.

6. A method for sequestering Aβ in the periphery comprising blood or blood components of an individual in need thereof, comprising:
a) administering an agent having binding affinity for amyloid beta (Aβ) in the periphery of the individual in need thereof;
b) sequestering Aβ in the periphery, thereby concomitantly decreasing Aβ levels in the brain of the individual.

7. The method according to claim 6, wherein the agent having binding affinity for amyloid beta (Aβ) is administered in the absence of immunomodulating agents or brain penetrance.

8. The method according to claim 6, wherein the individual is suffering from an amyloid beta (Aβ)-associated disease.

9. The method according to claim 8, wherein the amyloid beta (Aβ)-associated disease is selected from Alzheimer's disease, β-amyloid related problems of Down's syndrome, vascular dementia (cerebral amyloid angiopathy) and amyloidosis.

10. The method according to claim 6, wherein the agent having binding affinity for amyloid beta (Aβ) is selected from the group consisting of GM1 ganglioside, gelsolin, an Aβ imaging agent, a β-sheet breaker, a β-sheet formation inhibitor and a derivative of an amyloid beta (Aβ)-staining dye.

11. The method according to claim 10, wherein the derivative of the amyloid beta (Aβ)-staining dye is 1,4-bis(3-carboxy-4-hydroxyphenylethenyl)-benzene and 5,5′-{(1,1′biphenyl)-4,4′-diylbis(azo)}bis {2-hydroxybenzoic acid}disodium salt (chrysamine-G).

12. A method of monitoring the effectiveness of drug treatment of beta-amyloid related diseases, comprising:
(a) assessing levels of Aβ in the periphery of a recipient of the drug treatment; and
(b) determining an elevation of the levels of Aβ in the periphery of the recipient.

13. The method according to claim 12, wherein the levels (Aβ) are assessed by an assay that detects Aβ.

14. The method according to claim 13, wherein the assay is (i) a radioisotopic immunoassay or (ii) a non-isotopic immunoassay.

15. The method according to claim 14, wherein the non-isotopic immunoassay is selected from a fluorescent immunoassay, a chemiluminescent immunoassay, or an enzymatic immunoassay (ELISA).

16. The method according to claim 12, wherein the drug treatment comprises an agent having binding affinity for amyloid beta (Aβ).

17. The method according to claim 16, wherein the agent is selected from the group consisting of GM1 ganglioside, gelsolin, an Aβ imaging agent, a β-sheet breaker, a β-sheet formation inhibitor and a derivative of an amyloid beta (Aβ)-staining dye.

18. The method according to claim 17, wherein the derivative of the amyloid beta (Aβ)-staining dye is 1,4-bis(3-carboxy-4-hydroxyphenylethenyl)-benzene and 5,5′-{(1,1′biphenyl)-4,4′-diylbis(azo)}bis {2-hydroxybenzoic acid) disodium salt (chrysamine-G).

19. The method according to claim 12, wherein the monitoring occurs at about 1-25 days following administration of drug to the recipient.

20. The method according to claim 12, wherein the monitoring occurs at about 5-10 days following administration of drug to the recipient.

21. A method of treating amyloidosis in a subject, said method comprising administering in the periphery of said subject an amyloid beta (Aβ) binding agent, said agent selected from the group consisting of GM1 ganglioside, gelsolin, an Aβ imaging agent, a β-sheet breaker, a β-sheet formation inhibitor and a derivative of an amyloid beta (Aβ)-staining dye, for a time and under conditions suitable for the agent to bind amyloid beta (Aβ), sequester amyloid beta (Aβ) in the periphery and decrease amyloid beta (Aβ) levels in the brain of the individual.

22. The method according to claim 21, wherein the derivative of the amyloid beta (Aβ)-staining dye is 1,4-bis(3-carboxy-4-hydroxyphenylethenyl)-benzene and 5,5-′(1,1′biphenyl)-4,4′-diylbis(azo)}bis {2-hydroxybenzoic acid}disodium salt (chrysamine-G).
